(19) United States
(12) Patent Application Publication
(10) Pub. No.: US 2023/0019319 A1
(43) Pub. Date: Jan. 19, 2023

(54) LEAD INTEGRITY EVALUATION BASED ON IMPEDANCE VARIABILITY

(71) Applicant: Medtronic, Inc., Minneapolis, MN (US)

(72) Inventors: Adam K. Himes, Plymouth, MN (US); Charles D. Swerdlow, Los Angeles, CA (US); Scott A. Hoium, Burtrum, MN (US); Chad A. Bounds, Walnut Creek, CA (US)

(21) Appl. No.: 17/810,905

(22) Filed: Jul. 6, 2022

Related U.S. Application Data
(60) Provisional application No. 63/220,169, filed on Jul. 9, 2021.

(51) Int. Cl. A61N 1/372; A61N 1/08

(52) U.S. Cl. CPC A61N 1/37258; A61N 1/08; A61N 2001/083

(57) ABSTRACT

A method comprises acquiring a set of measurements of impedance of an implantable medical lead, determining a metric of variability of the set of impedance measurements, determining that the metric of variability satisfies a criterion, and generating a lead integrity alert in response to the metric of variability satisfying the criterion.

This application claims the benefit of U.S. Provisional Patent Application Ser. No. 63/220,169, filed Jul. 9, 2021, the entire content of which is incorporated herein by reference.

### TECHNICAL FIELD

The disclosure relates generally to medical devices and, more particularly, techniques for evaluating the integrity of implantable medical leads.

### BACKGROUND

A variety of implantable medical devices for delivering a therapy and/or monitoring a physiological condition have been clinically implanted or proposed for clinical implantation in patients. Implantable medical devices may deliver electrical or drug therapy and/or monitor conditions associated with the heart, muscle, nerve, brain, stomach or other organs or tissue. Some implantable medical devices may employ one or more elongated electrical leads carrying therapy electrodes, sense electrodes, and/or other sensors. Implantable medical leads may be configured to allow electrodes or other sensors to be positioned at desired locations for delivery of therapy or sensing. For example, electrodes or sensors may be carried at a distal portion of a lead. A proximal portion of the lead may be coupled to an implantable medical device housing, which may contain circuitry such as therapy delivery and/or sensing circuitry.

Implantable medical devices, such as cardiac pacemakers or implantable cardioverter-defibrillators, for example, provide electrical therapy to the heart via electrodes carried by one or more implantable medical leads. The electrical therapy may include signals such as pacing pulses or shocks for cardioversion or defibrillation. In some cases, an implantable medical device may sense intrinsic depolarizations of the heart, and control delivery of therapy signals to the heart based on the sensed depolarizations. Upon detection of an abnormal rhythm, such as bradycardia, tachycardia, or fibrillation, an appropriate electrical therapy signal or signals may be delivered to restore or maintain a more normal rhythm.

Implantable medical leads typically include a lead body containing one or more elongated electrical conductors that extend through the lead body from a connector assembly provided at a proximal lead end to one or more electrodes located at the distal lead end or elsewhere along the length of the lead body. The conductors connect therapy delivery and/or sensing circuitry within an associated implantable medical device housing to respective electrodes or sensors. Some electrodes may be used for both therapy delivery and sensing. Each electrical conductor is typically electrically isolated from other electrical conductors and is encased within an outer sheath that electrically insulates the lead conductors from body tissue and fluids.

When implanted, implantable medical leads may be subjected to forces and/or conditions that may negatively affect the integrity of the lead. Cardiac implantable medical leads, for example, tend to be continuously flexed by the beating of the heart. Other stresses may be applied to the implantable medical lead during implantation or lead repositioning. Patient movement can cause the route traversed by the implantable medical lead to be constricted or otherwise altered, causing stresses on the lead. Such stresses may lead to fracture of one or more conductors of the lead, or externalization of conductors from the insulative body of the implantable medical lead. Additionally, the electrical connection between implantable medical device connector elements and the lead connector elements can be intermittently or continuously disrupted. Connection mechanisms, such as set screws, may be insufficiently tightened at the time of implantation, followed by a gradual loosening of the connection. Also, lead pins may not be completely inserted. In some cases, changes in lead conductors or connections may result in intermittent or continuous changes in lead impedance.

Short circuits, open circuits or significant changes in impedance may be referred to, in general, as lead related conditions. In the case of cardiac implantable medical leads, sensing of an intrinsic heart rhythm through a lead can be altered by lead related conditions. Structural modifications to leads, conductors or electrodes may alter sensing integrity. Furthermore, impedance changes in the electrical path due to lead related conditions may affect sensing and therapy integrity for pacing, cardioversion, or defibrillation. For example, in some rare instances, a lead related issue may result in inappropriate detection of a ventricular fibrillation episode and the resultant delivery of high-voltage anti-tachyarrhythmia therapy.

### SUMMARY

Some lead integrity evaluation techniques include monitoring one or more of lead impedance, oversensing (e.g., as indicated by non-physiologic R-R intervals and/or non-sustained tachyarrhythmias (NSTs)), saturation, clipping, or other changes in the amplitude of the cardiac electrogram (EGM) signal. Existing impedance-based lead integrity diagnostics may have an impedance resolution on the order of tens of ohms, and a temporal resolution on the order of one measurement every N hours. Existing impedance-based lead integrity diagnostics may also be confounded by changes in the impedance of patient tissue or fluid. The criterion for detecting a fracture using existing impedance-based lead integrity diagnostics may be an impedance threshold on the order of hundreds or thousands of ohms. In contrast, partial fracture of a lead conductor may result in a change of less than an ohm. Consequently, existing impedance-based lead integrity diagnostics may be unable to detect partial conductor fractures, which may cause sensing and therapy integrity issues, and may ultimately become complete fractures.

This disclosure describes techniques for evaluating the integrity of implantable medical leads based on variability (e.g., standard deviation) of impedance measurements. In some examples, the techniques include determining the variability of a sequence of impedance measurements made at a rate significantly higher than existing lead integrity impedance measurements, such as a number of samples per second. In some examples, the techniques include acquiring X (e.g., 500) samples of impedance at Y (e.g., 65) samples per second, with a resolution of less than or equal to Z (e.g., 0.1 ohm). The techniques of this disclosure may advantageously enable earlier detection of fracture of conductors of implantable medical leads and/or detection of partial fracture of conductors of implantable medical leads.

In one example, a method comprises: acquiring a set of measurements of impedance of an implantable medical lead; determining a metric of variability of the set of impedance measurements; determining that the metric of variability satisfies a criterion; and generating a lead integrity alert in response to determining that the metric of variability satisfies the criterion.

In another example, a system comprises an implantable medical device configured to measure impedance of an implantable medical lead coupled to the implantable medical device; and processing circuitry. The processing circuitry is configured to: acquire a set of measurements of impedance of the implantable medical lead by the implantable medical device; determine a metric of variability of the set of impedance measurements; determine that the metric of variability satisfies a criterion; and generate a lead integrity alert in response to determining that the metric of variability satisfies the criterion.

In another example, a non-transitory computer-readable storage medium comprises instructions that, when executing by processing circuitry, cause the processing circuitry to: acquire a set of measurements of impedance of the implantable medical lead by the implantable medical device; determine a metric of variability of the set of impedance measurements; determine that the metric of variability satisfies a criterion; and generate a lead integrity alert in response to determining that the metric of variability satisfies the criterion.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1 is an example schematic diagram of an implantable medical device system configured to evaluate the integrity of an implantable medical lead based on impedance variability.

FIG. 2 is a functional block diagram of an example implantable medical device configured to evaluate the integrity of an implantable medical lead based on impedance variability.

FIG. 3 is a functional block diagram of an example external device configured to communicate with an implantable medical device.

FIG. 4 is a functional block diagram illustrating an example system that includes external computing devices, such as a server and one or more other computing devices, that are coupled to the implantable and external devices shown in FIG. 1 via a network.

FIG. 5 is a flow diagram illustrating an example method for evaluating the integrity of an implantable medical lead based on impedance variability.

FIG. 6 is a conceptual cross-sectional diagram illustrating an example implantable medical lead.

FIGS. 7-25 are diagrams illustrating data collected during experiments using IMDs and implantable medical leads as illustrated and described with respect to FIGS. 1-6.

### DETAILED DESCRIPTION

As described above, methods, devices, and systems for evaluating the integrity of an implantable medical lead based on impedance variability are described in this disclosure. In the following description, references are made to illustrative examples. It is understood that other examples may be utilized without departing from the scope of the disclosure.

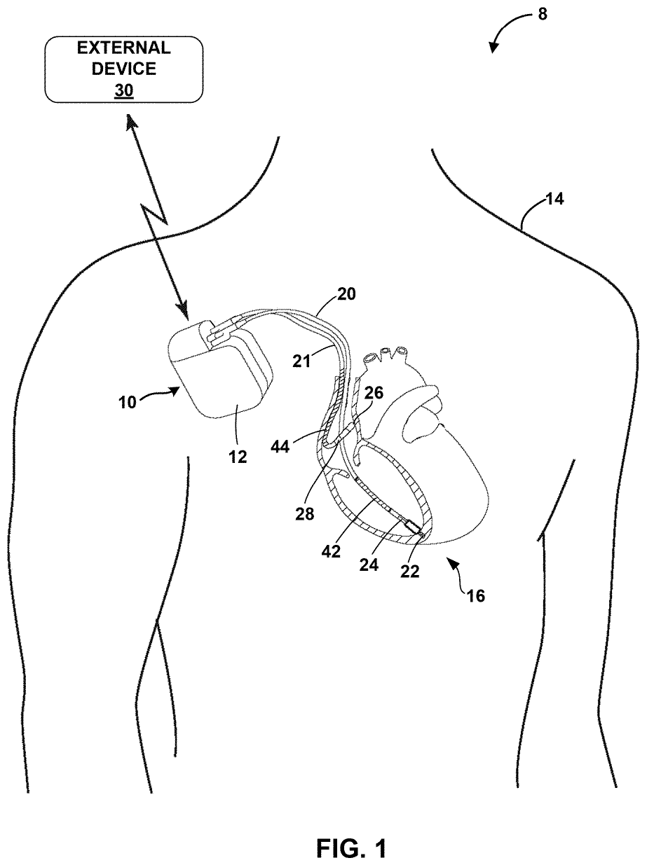

FIG. 1 is an example schematic diagram of an implantable medical device system configured to evaluate the integrity of an implantable medical lead based on impedance variability. As illustrated in FIG. 1, a medical device system 8 for sensing cardiac events (e.g., P-waves and R-waves) and detecting tachyarrhythmia episodes, as well as evaluate the integrity of an implantable medical lead based on impedance variability, may include an implantable medical device (IMD) 10, a first (ventricular) implantable medical lead 20 and a second (atrial) implantable medical lead 21. In one example, IMD 10 may be an implantable cardioverter-defibrillator (ICD) capable of delivering pacing, cardioversion, and defibrillation therapy to the heart 16 of a patient 14. In other examples, IMD 10 may be a pacemaker capable of delivering pacing therapy, including anti-tachycardia pacing (ATP) to the patient, but need not include the capability of delivering cardioversion or defibrillation therapies.

Ventricular lead 20 and atrial lead 21 are electrically coupled to IMD 10 and extend into heart 16. Ventricular lead 20 includes electrodes 22 and 24 shown positioned on the lead in the right ventricle (RV) of heart 16 for sensing ventricular EGM signals and pacing in the RV. Atrial lead 21 includes electrodes 26 and 28 positioned on the lead in the right atrium (RA) of heart 16 for sensing atrial EGM signals and pacing in the RA.

In the example of FIG. 1, ventricular lead 20 additionally carries a high voltage coil electrode 42, and atrial lead 21 carries a high voltage coil electrode 44, used to deliver cardioversion and defibrillation shock pulses. In other examples, ventricular lead 20 may carry both of high voltage coil electrodes 42 and 44, or may carry a high voltage coil electrode in addition to those illustrated in the example of FIG. 1. Both ventricular lead 20 and atrial lead 21 may be used to acquire cardiac EGM signals from patient 14 and to deliver therapy in response to the acquired data. Medical device system 8 is shown as a dual chamber ICD including atrial lead 21 and ventricular lead 20, but in some embodiments, system 8 may be a dual or multi-chamber system including a coronary sinus lead extending into the right atrium, through the coronary sinus and into a cardiac vein to position electrodes along the left ventricle (LV) for sensing LV EGM signals and delivering pacing pulses to the LV. In some examples, system 8 may be a single chamber system, or otherwise not include atrial lead 21.

Implantable medical device circuitry configured for performing the methods described herein and an associated battery or batteries are housed within a sealed housing 12 of IMD 10. Housing 12 may be conductive so as to serve as an electrode for use as an indifferent electrode during pacing or sensing or as an active electrode during defibrillation. As such, housing 12 is also referred to herein as “housing electrode” 12. In other examples, an indifferent electrode may be separate from housing 12 and placed elsewhere on IMD 10, such as in the header.

Implantable medical leads 20, 21 may include respective conductors connecting each of electrodes 22, 24, 26, 28, 42, and 44 to a connector assembly at the proximal end of the respective one of leads 20, 21, and thereby to the circuitry within housing 12 of IMD 10. Implantable medical leads 20, 21 may be subjected to forces and/or conditions (e.g., due to the beating of heart 16, motion of patient 14, or during implantation) that may negatively affect the integrity of the lead. Such forces may lead to fracture of one or more conductors of implantable medical leads 20, 21.

EGM signal data, cardiac rhythm episode data, and lead integrity data acquired by IMD 10 can be transmitted to an external device 30. External device 30 may be a computing device, e.g., used in a home, ambulatory, clinic, or hospital setting, to wirelessly communicate with IMD 10. External device 30 may be coupled to a remote patient monitoring system, such as Carelink®, available from Medtronic plc, of Dublin, Ireland. External device 30 may be, as examples, a programmer, external monitor, gateway, or consumer device (e.g., smart phone).

External device 30 may be used to program commands or operating parameters into IMD 10 for controlling IMD function, e.g., when configured as a programmer for IMD 10. External device 30 may be used to interrogate IMD 10 to retrieve data, including device operational data as well as physiological data accumulated in IMD memory. The interrogation may be automatic, e.g., according to a schedule, or in response to a remote or local user command. Programmers, external monitors, and consumer devices are examples of external devices 30 that may be used to interrogate IMD 10. Examples of communication techniques used by IMD 10 and external device 30 include radiofrequency (RF) telemetry, which may be an RF link established via Bluetooth, WiFi, or medical implant communication service (MICS).

One or more components of system 8 may evaluate the integrity of one or both of implantable medical leads 20, 21 based on variability of measured impedances of the implantable medical leads. For example, IMD 10 may measure a plurality of impedances (e.g., a plurality of impedance samples over a measurement period) of each of one or more paths, each path including one or more conductors of implantable medical leads 20, 21. Processing circuitry of system 8 (e.g., of IMD 10, external device 30, and/or another computing device not shown in FIG. 1) may determine a metric of the variability of the measure impedances, such as a standard deviation of the measured impedance. The processing circuitry may compare the metric of variability to a threshold or other criterion, and take one or more actions based on satisfaction of the criterion. For example, the processing circuitry may generate a lead integrity alert in response to the metric of variability meeting or exceeding a threshold. The lead integrity alert may be presented to a user via external device 30 or another computing device. In some examples, the processing circuitry may additionally or alternatively change a sensing or therapy vector, cause more frequent performances of the lead impedance measurements (more frequent measurement periods), and/or cause performance of other lead integrity diagnostics.

Although described herein primarily with respect to cardiac devices and intracardiac implantable medical leads, the techniques of this disclosure may be implemented in systems that additionally or alternatively include implantable medical leads implanted in other locations, such as an extracardiac location, epidural location, cranial location, gastric location, pelvic location, or within a limb. The implantable medical leads may be coupled to medical devices configured to provide sensing or therapy for cardiac conditions, neurological conditions, or other conditions.

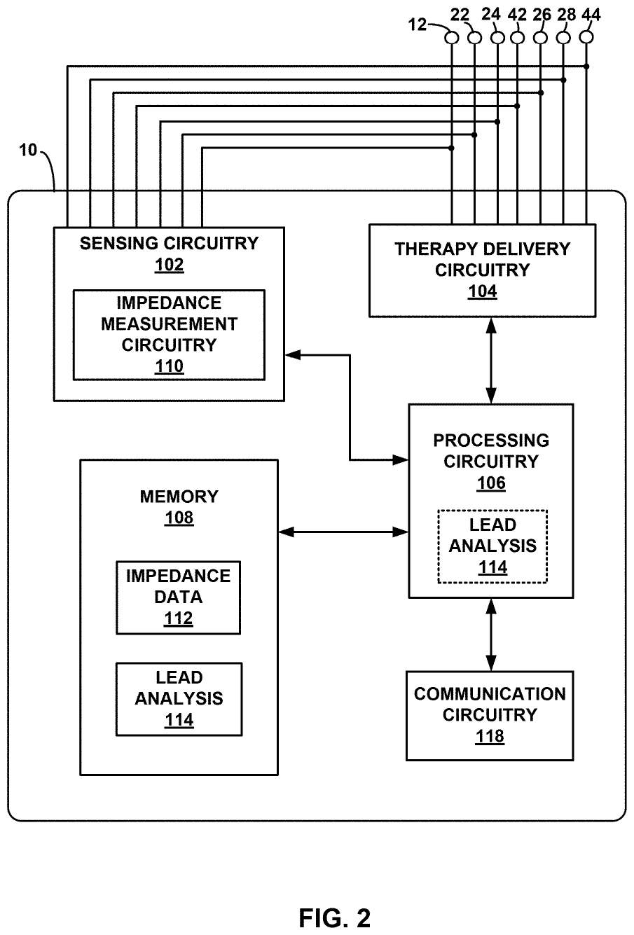

FIG. 2 is a functional block diagram of an example configuration of IMD 10. In the example illustrated by FIG. 2, IMD 10 includes sensing circuitry 102, therapy delivery circuitry 104, processing circuitry 106, associated memory 108, and communication circuitry 118.

Processing circuitry 106 may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 106 may include multiple components, such as any combination of one or more microprocessors, one or more DSPs, one or more ASICs, or one or more FPGAs, as well as other discrete or integrated logic circuitry, and/or analog circuitry.

Memory 108 may store program instructions, which may include one or more program modules, which are executable by processing circuitry 106. When executed by processing circuitry 106, such program instructions may cause processing circuitry 106 and IMD 10 to provide the functionality ascribed to them herein. The program instructions may be embodied in software, firmware and/or RAMware. Memory 108 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Sensing circuitry 102 is configured to receive cardiac electrical signals from selected combinations of two or more of electrodes 22, 24, 26, 28, 42 and 44 carried by the ventricular lead 20 and atrial lead 21, along with housing electrode 12. Sensing circuitry 102 is configured to sense cardiac events attendant to the depolarization of myocardial tissue, e.g. P-waves and R-waves. Sensing circuitry 102 may include switching circuitry for selectively coupling electrodes 12, 22, 24, 26, 28, 42, 44 to sensing circuitry 102 in order to monitor electrical activity of heart 16. In other examples, not shown in FIG. 2, sensing circuitry 102 may receive cardiac electrical signals from other electrodes such as one or more LV electrodes, as described above in relation to FIG. 1. The switching circuitry may include a switch array, switch matrix, multiplexer, or any other type of switching device suitable to selectively couple one or more of the electrodes to sensing circuitry 102. In some examples, processing circuitry 106 selects the electrodes to function as sense electrodes, or the sensing vector, via the switching circuitry within sensing circuitry 102.

Sensing circuitry 102 may include multiple sensing channels, each of which may be selectively coupled to respective combinations of electrodes 12, 22, 24, 26, 28, 42, 44 to detect electrical activity of a particular chamber of heart 16, e.g., an atrial sensing channel and one or more ventricular sensing channels. Each sensing channel may be configured to amplify, filter, and rectify the cardiac electrical signal received from selected electrodes coupled to the respective sensing channel to detect cardiac events, e.g., P-waves and/or R-waves. For example, each sensing channel may include one or more filters and amplifiers for filtering and amplifying a signal received from a selected pair of electrodes. The resulting cardiac electrical signal may be passed to cardiac event detection circuitry that detects a cardiac event when the cardiac electrical signal crosses a sensing threshold. The cardiac event detection circuitry may include a rectifier, filter and/or amplifier, a sense amplifier, comparator, and/or analog-to-digital converter.

Sensing circuitry 102 outputs an indication to processing circuitry 106 in response to sensing of a cardiac event, in the respective chamber of heart 16 (e.g., detected P-waves or R-waves). In this manner, processing circuitry 106 may receive detected cardiac event signals corresponding to the occurrence of detected R-waves and P-waves in the respective chambers of heart 16. Indications of detected R-waves and P-waves may be used for detecting ventricular and/or atrial tachyarrhythmia episodes, e.g., ventricular or atrial fibrillation episodes. Sensing circuitry 102 may also pass one or more digitized EGM signals to processing circuitry 106 for analysis, e.g., for use in cardiac rhythm discrimination.

As illustrated in FIG. 2, sensing circuitry 102 may include impedance measurement circuitry 110 configured to make measurements of the impedance of paths including conductors respectively coupled to electrodes 22, 24, 26, 28, 42, 44. Each path may include two of electrodes 22, 24, 26, 28, 42, 44, or one of the electrodes 22, 24, 26, 28, 42, 44 in combination with housing electrode 12. Impedance measurement circuitry 110 may include circuitry configured to generate and deliver a current or voltage signal via the path (e.g., a pulse or sinusoidal signal), measure the resulting voltage or current, and determine impedance based on the measured voltage or current. The resulting voltage or current may be measured at dc or a variety of frequencies. Impedance measurement circuitry 110 may include capacitors, charge pumps, transistors, or the like for generating the signal, and sample and hold circuitry for measuring the resulting signal. Processing circuitry 106 may store the measured impedance values as impedance data 112 in memory 108.

Memory 108 may also store a lead analysis module 114. Lead analysis module 114 may be a software, firmware, or RAMware module executable by processing circuitry 106 to cause processing circuitry 106 to provide functionality related to evaluating the integrity of implantable medical leads 20, 21. Such functionality may include determining a metric of variability of impedance measurements, and comparing the metric to a criterion, as described herein. Processing circuitry 106 may load lead analysis module 114 from memory 108 (shown by the dotted lead analysis module 114 within processing circuitry 106) and execute the loaded lead analysis module 114 in response to an event, such as oversensing or a patient posture or activity satisfying one or more criteria. In other examples, processing circuitry 106 may execute lead analysis module 114 periodically, e.g., according to a schedule (e.g., N executions per day), or substantially continuously, throughout the operation of IMD 10. The techniques of this disclosure may be particularly useful in evaluating the integrity of conductors connected to defibrillation electrodes 42 and 44, which are not typically used for cardiac EGM sensing and therefore not easily able to be evaluated using oversensing or other diagnostics related to EGM sensing.

Processing circuitry 106 may control therapy delivery circuitry 104 to deliver electrical therapy, e.g., cardiac pacing, anti-tachyarrhythmia therapy, or cardioversion or defibrillation shock pulses, to heart 16 according to therapy parameters stored in memory 108. Therapy delivery circuitry 104 is electrically coupled to electrodes 12, 22, 24, 26, 28, 42, 44, and is configured to generate and deliver electrical therapy to heart 16 via selected combinations of electrodes 12, 22, 24, 26, 28, 42, 44. Therapy delivery circuit 104 may include charging circuitry, one or more charge storage devices, such as one or more high voltage capacitors and/or one or more low voltage capacitors, and switching circuitry that controls when the capacitor(s) are discharged to selected combinations of electrodes 12, 22, 24, 26, 28, 42, 44. Charging of capacitors to a programmed pulse amplitude and discharging of the capacitors for a programmed pulse width may be performed by therapy delivery circuit 104 according to control signals received from processing circuitry 106.

Memory 108 stores intervals, counters, or other data used by processing circuitry 106 to control the delivery of pacing pulses by therapy delivery circuitry 104. Such data may include intervals and counters used by processing circuitry 106 to control the delivery of pacing pulses to heart 16. The intervals and/or counters are, in some examples, used by processing circuitry 106 to control the timing of delivery of pacing pulses relative to an intrinsic or paced event in another chamber. Memory 108 also stores intervals for controlling cardiac sensing functions such as blanking intervals and refractory sensing intervals and counters for counting sensed events for detecting cardiac rhythm episodes. Events sensed by sense amplifiers included in sensing circuitry 102 are identified in part based on their occurrence outside a blanking interval and inside or outside of a refractory sensing interval. Events that occur within predetermined interval ranges are counted for detecting cardiac rhythms. According to embodiments described herein, sensing circuitry 102, therapy circuitry 104, memory 108, and processing circuitry 106 are configured to use timers and counters for measuring sensed event intervals and determining event patterns for use in detecting possible ventricular lead dislodgement.

Communication circuitry 118 is used to communicate with external device 30, for transmitting data accumulated by IMD 10 and for receiving interrogation and programming commands from external device 30. Under the control of processing circuitry 106, telemetry circuitry 118 may transmit an alert to notify a clinician and/or the patient that IMD 10 has detected a possible lead integrity issue. This alert enables the clinician to perform additional testing to confirm the issue and to intervene if necessary to select different sensing or therapy vectors or replace the lead. In other embodiments, IMD 10 may be equipped with alert circuitry configured to emit a sensory alert perceptible by the patient, e.g. a vibration or an audible tone, under the control of processing circuitry 106 to alert the patient to the possibility of a lead integrity issue.

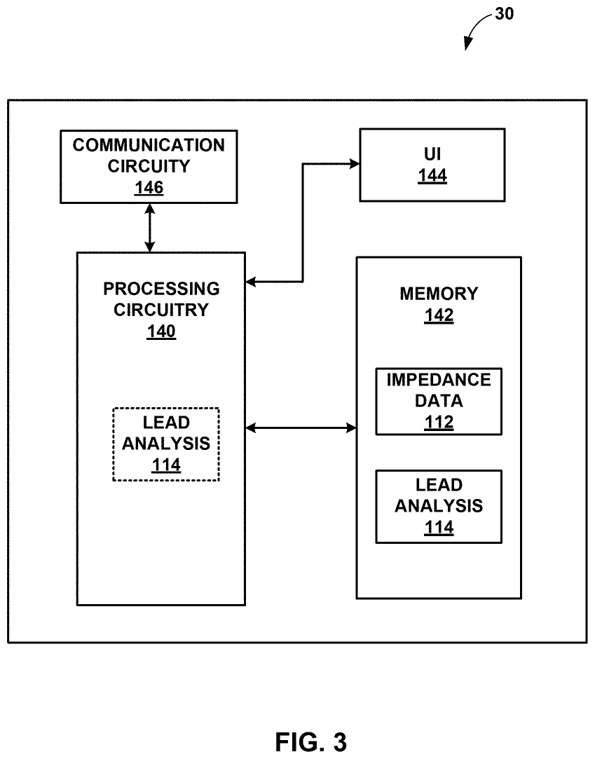

FIG. 3 is a functional block diagram of an example configuration of external device 30. In the example of FIG. 3, external device 30 includes processing circuitry 140, memory 142, user interface (UI) 144, and communication circuitry 146. External device 30 may be a dedicated hardware device with dedicated software for the programming and/or interrogation of IMD 10. Alternatively, external device 30 may be an off-the-shelf computing device, e.g., running an application that enables external device 30 to program and/or interrogate IMD 10.

In some examples, a user uses external device 30 to select or program values for operational parameters of IMD 10, e.g., for cardiac sensing, therapy delivery, and lead integrity evaluation. In some examples, a user uses external device 30 to receive data collected by IMD 10, such as impedance data 112 or other operational and performance data of IMD 10. The user may also receive lead integrity alerts provided by IMD 10, or data regarding modifications to sensing or therapy made by IMD 10 in response to detecting lead integrity issues. The user may interact with external device 30 via UI 144, which may include a display to present a graphical user interface to a user, and a keypad, touchpad or another mechanism for receiving input from a user. External device 30 may communicate wirelessly with IMD 10 using communication circuitry 146, which may be configured for wireless communication with communication circuitry 118 of IMD 10.

Processing circuitry 140 may include any combination of integrated circuitry, discrete logic circuity, analog circuitry, such as one or more microprocessors, DSPs, ASICs, or FPGAs. In some examples, processing circuitry 106 may include multiple components, such as any combination of one or more microprocessors, one or more DSPs, one or more ASICs, or one or more FPGAs, as well as other discrete or integrated logic circuitry, and/or analog circuitry.

Memory 142 may store program instructions, which may include one or more program modules, which are executable by processing circuitry 140. When executed by processing circuitry 140, such program instructions may cause processing circuitry 140 and external device 30 to provide the functionality ascribed to them herein. The program instructions may be embodied in software and/or firmware. Memory 142 may include any volatile, non-volatile, magnetic, optical, or electrical media, such as a RAM, ROM, NVRAM, EEPROM, flash memory, or any other digital media.

In some examples, processing circuitry 140 of external device 30 may be configured to provide some or all of the functionality ascribed to processing circuitry 106 of IMD 10 herein. For example, processing circuitry 140 may determine a metric of variability of impedance measurements, and compare the metric to a criterion. Based on satisfaction of the criterion, processing circuitry 140 may provide an alert to a user, e.g., via UI 144. In some examples, the lead integrity evaluation functionality may be provided by lead analysis module 114, which may a software module stored in memory 142, and loaded and executed by processing circuitry 140 (as illustrated by the dotted outline of lead analysis module 114 within processing circuitry 140), e.g., in response to a command from the user.

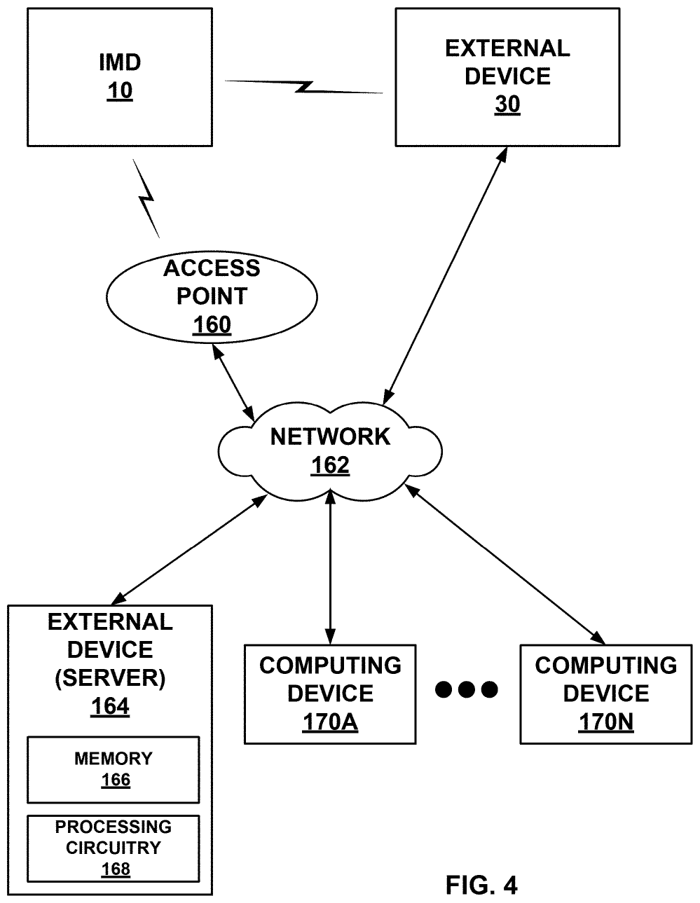

FIG. 4 is a functional block diagram illustrating an example system that includes external computing devices, such as a server 164 and one or more other computing devices 170A-170N, that are coupled to IMD 10 and external device 30 via a network 162. In this example, IMD 10 may use its telemetry module 118 to, e.g., at different times and/or in different locations or settings, communicate with external device 30 via a first wireless connection, and to communicate with an access point 160 via a second wireless connection. In the example of FIG. 4, access point 160, external device 30, server 164, and computing devices 170A-170N are interconnected, and able to communicate with each other, through network 162.

Access point 160 may comprise a device that connects to network 162 via any of a variety of connections, such as telephone dial-up, digital subscriber line (DSL), or cable modem connections. In other examples, access point 160 may be coupled to network 162 through different forms of connections, including wired or wireless connections. In some examples, access point 160 may be co-located with patient 14. Access point 160 may interrogate IMD 10, e.g., periodically or in response to a command from patient 14 or network 162, to retrieve impedance data 112 or other operational data from IMD 10. Access point 160 may provide the retrieved data to server 164 via network 162.

In some cases, server 164 may be configured to provide a secure storage site for data that has been collected from IMD 10 and/or external device 30, such as the Internet. In some cases, server 164 may assemble data in web pages or other documents for viewing by trained professionals, such as clinicians, via computing devices 170A-170N. The illustrated system of FIG. 4 may be implemented, in some aspects, with general network technology and functionality similar to that provided by the Medtronic CareLink® Network developed by Medtronic plc, of Dublin, Ireland.

In some examples, one or more of access point 160, server 164, or computing devices 170 may be configured to perform, e.g., may include processing circuitry configured to perform, some or all of the techniques described herein relating to evaluating integrity of an implantable medical lead. In the example of FIG. 4, server 164 includes a memory 166 to store EGM data received from IMD 10, and processing circuitry 168, which may be configured to provide some or all of the functionality ascribed to processing circuitry 106 of IMD 10 herein. For example, processing circuitry 168 may determine a metric of variability of impedance measurements, compare the metric to a criterion, and provide a lead integrity alert to a user, e.g., via external device 30 or one of computing devices 170.

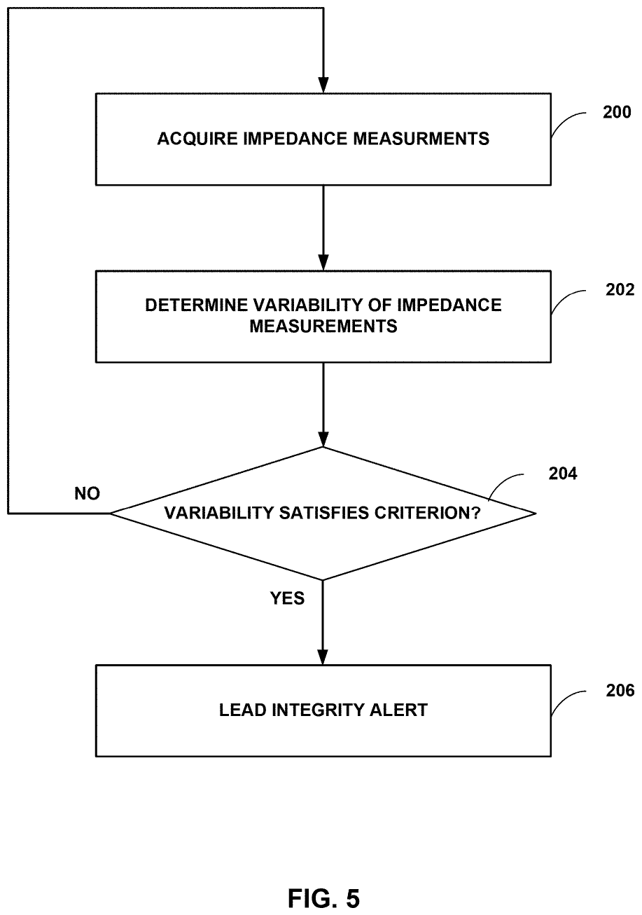

FIG. 5 is a flow diagram illustrating an example method for evaluating the integrity of an implantable medical lead based on impedance variability. The example method of FIG. 5 is described as being performed by IMD 10, including processing circuitry 106. In some examples, portions of the example method of FIG. 5 may be performed by processing circuitry 140 of external device 30 or processing circuitry 168 of server 164, alone or in combination with processing circuitry 30. The example method of FIG. 5 may be performed for each of a plurality of paths, e.g., including a respective electrode or electrode pair. The example method of FIG. 5 may be performed repeatedly, e.g., according to a schedule or as triggered by an event.

According to the example of FIG. 5, processing circuitry 140 acquires impedance measurements (200). In some examples, processing circuitry 140 controls impedance measurement circuitry 110 to make the impedance measurements. For example, processing circuitry 140 may control impedance measurement circuitry 110 to, for each of one or more paths, sample the impedance X (e.g., 500) times at a rate of Y (e.g., 65) samples per second. Impedance measurement circuitry 110 may be configured to measure impedance with a resolution less than Z (e.g., 0.1 ohm).

Processing circuitry 140 determines a metric of the variability of the impedance measurements (e.g., of the 500 samples for a given path) (202). Example metrics of variability include standard deviation, range, and variance. Processing circuitry 140 determines whether the metric of variability satisfies a criterion, such as meeting or exceeding a threshold (e.g., 0.5 ohms) (204). If the criterion is not satisfied (NO of 204), processing circuitry 140 may acquire additional impedance measurements (200). If the criterion is satisfied (YES of 204), processing circuitry 140 may generate a lead integrity alert (206), which may be communicated to external device 30 and/or server 164. The lead integrity alert may indicate the value of the impedance variability metric. The lead integrity alert may indicate a fracture, partial fracture, or anticipated fracture of a conductor associated with a particular electrode or pair of electrodes in the path tested. Although primarily described with respect to fracture, the techniques described herein may be used to detect other lead-related issues, such as externalization of a conductor, a short circuit, an open circuit, or an issue with the lead-IMD connection, such as incomplete connector pin insertion.

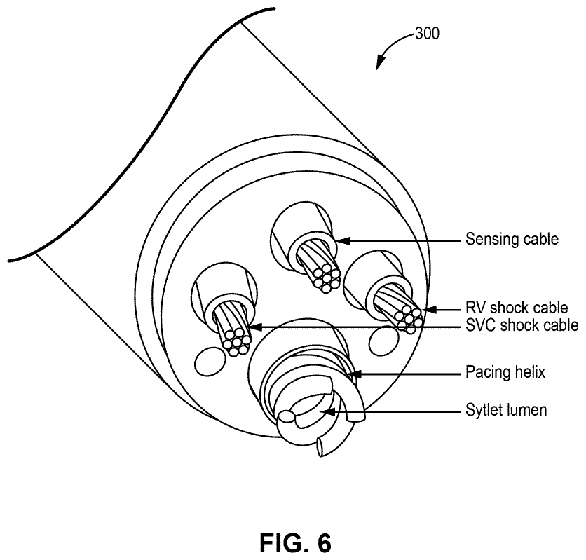

FIG. 6 is a conceptual cross-sectional diagram illustrating an example implantable medical lead (300). Implantable medical lead 300 may be a ventricular lead, and may be coupled to IMD 10 to function substantially as described with respect to implantable medical lead 20. FIG. 6 illustrates the conductors within implantable medical lead 300. Each of the conductors is disposed within a respective insulator which together extend though the insulative body of the implantable medical lead.

As illustrated in FIG. 6 implantable medical lead 300 includes a pacing helix, which may be connected to a tip electrode, such as electrode 22 (FIG. 1). The pacing helix defines a stylet lumen. Implantable medical lead 300 also includes a sensing cable, which may be connected to a ring electrode, such as electrode 24 (FIG. 1). Implantable medical 300 also includes an RV shock cable coupled to an RV shock electrode, such as electrode 42, and an SVC shock cable connected to an SVC shock electrode, which may be provided as an alternative to electrode 44 on implantable medical lead 21.

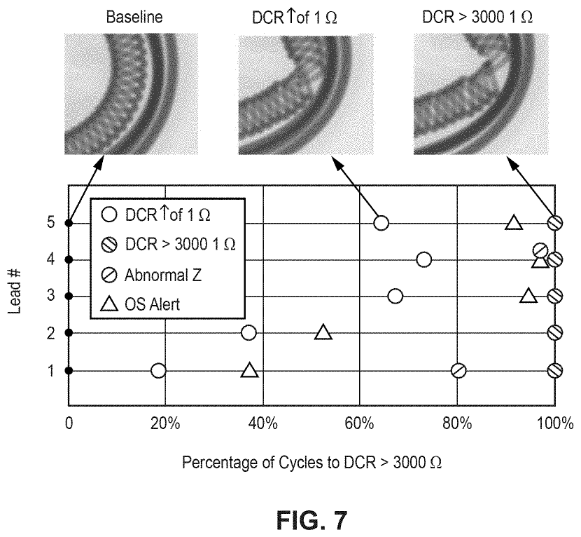

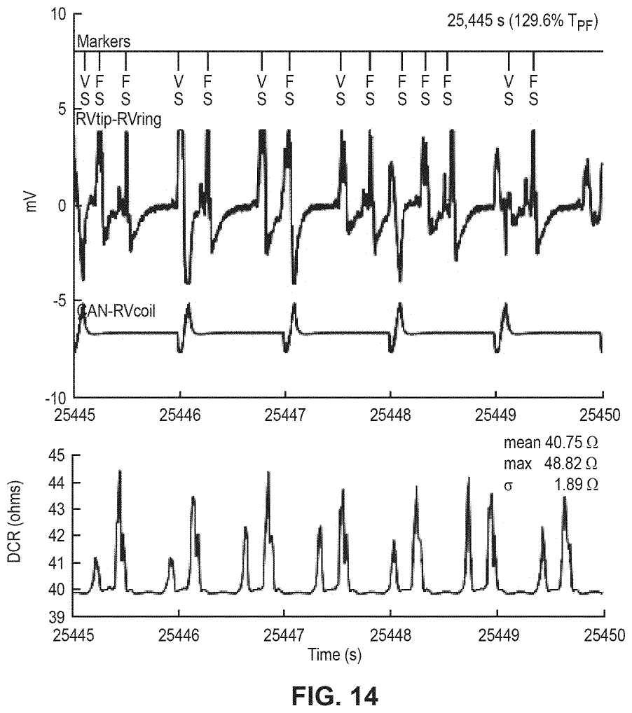

FIGS. 7-25 are diagrams illustrating data collected during experiments using IMDs and implantable medical leads as illustrated and described with respect to FIGS. 1-6.

The relationship between developing conductor fracture (Fx) and ICD diagnostics (such as oversensing and impedance diagnostics) is unknown because bench tests usually are performed on isolated lead segments, not intact leads connected to ICD generators. Experiments were performed to characterize this relationship.

In one experiment, accelerated, cyclic-bending tests were performed on 5 Medtronic Quattro™ leads connected to ICD generators and 500Ω pacing loads. A simulated pace/sense electrogram (EGM) was input and direct-current resistance (DCR) was measured with precision ±0.1Ω. DCR and EGM were recorded continuously. The ICD stored diagnostics and real-time EGMs every simulated day. An abnormal ICD diagnostic was defined as ≥75% increase in pacing impedance (Z) from baseline or an oversensing (OS) alert (≥30 ventricular intervals ≤130 milliseconds (ms) and ≥2 non-sustained tachycardia episodes).

The upper panel of FIG. 7 illustrates high-resolution radiographs at a 1Ω increase in DCR and at DCR>3000Ω. The lower panel of FIG. 7 illustrates results as median percentage of total cycles to the test's end at DCR>3000Ω.

In bench testing, DCR is highly-sensitive for fracture. Clinically, lead-monitoring oversensing alerts are more sensitive than impedance alerts.

In a further experiment, bending tests were performed on leads connected to ICD generators, in an electrolyte bath with simulated ECG input. EGMs were telemetered continuously; DCR was recorded every 3 min from the helical tip conductor. We defined partial helix fracture as DCR standard deviation ≥0.5Ω; complete fracture was DCR >3000Ω. We tested 12 leads to partial fracture and 9 leads to complete fracture. Results are reported as medians of multiples of time to partial fracture (T_{PF}).

Baseline s_{DCR} was ≤0.05Ω. In 9 complete tests, median T_{PF} and time to complete fracture were 334 and 580 minutes, respectively. Oversensing first occurred at 1.00 T_{PF}. The oversensing alert triggered at 1.13 T_{PFx}, before ICD-detected VF in 8 leads (p=0.006). The relative-impedance and impedance-threshold alerts triggered only at complete fracture (1.62 T_{PF}, p=0.006 vs. oversensing alert), after ICD-detected VF in 7 leads. Early fractures caused a DCR spike (median 4Ω) with each bending cycle and corresponding cyclical oversensed EGM. When bending stopped, EGMs normalized in all leads. Radiographs confirmed partial (n=5) and complete (n=9) fracture. In partial fracture conductor fracture, initial oversensing correlates with small, bending-induced DCR increases. This supports make-break potentials as the cause of fracture-induced oversensing. In contrast, clinical impedance alerts correlate with complete fracture.

Despite improvements in design and testing, transvenous right-ventricular (RV) defibrillation leads may fail during clinical service. Most failures involve pace-sense components, placing patients at risk for inappropriate shocks and loss of pacing. Implantable-cardioverter defibrillator (ICD) systems monitor for conductor fracture using pacing impedance and measures of oversensed non-physiologic signals. In in-vitro fatigue testing of conductor segments, small changes in pacing impedance are highly-sensitive for conductor fracture. In clinical practice, lead-monitoring alerts that measure oversensing are more sensitive than those that only use impedance. The temporal relationship between oversensing and impedance changes is unknown in leads with developing conductor fractures. To characterize this relationship, accelerated, cyclic-bending tests of a defibrillation lead placed in a saline bath and connected to an ICD generator were performed. In addition to tracking the time course of impedance and sensing changes, they were correlated with structural changes in the fractured conductor.

The experimental apparatus included an ICD system comprising a Medtronic Cobalt™ generator attached to a 65 centimeter (cm), Medtronic Sprint Quattro™ model 6947 right-ventricular (RV), dual-coil lead. Implantable medical lead 300 of FIG. 6 is an example of such as lead. The multi-lumen lead has a 4-filar, helical conducting coil (helix) to the distal (tip) pace-sense electrode, a cable to the ring pace-sense electrode, and proximal and distal defibrillation coils. The number of intervals to detect ventricular fibrillation (VF) was set to 30/40 with a VF detection interval of 320 ms. The ICD system was connected for electrical monitoring, and the lead was connected to a mechanical fixture for bending.

Medtronic ICDs measure pacing impedance every 6 hours and alert for values outside a programmable range, nominally 200-2000Ω. In this study, most tests lasted less than 12 hours, producing only 2 impedance measurements. Thus, impedance was estimated from direct current resistance (DCR), which was measured frequently as described below.

The Lead Integrity Alert™ (LIA), is comprised of both oversensing and impedance components. The two oversensing components are a count of at least 30 non-physiologic short ventricular intervals ≤130 ms within 3 days (Sensing Integrity Count™, SIC) and occurrence of at least 2 rapid non-sustained tachycardia episodes(<220 ms, NST) in 60 days. The relative-impedance component requires an abrupt change relative to a 13-day baseline (75% increase or 50% decrease). LIA is triggered when threshold criteria are satisfied for any two components. The RV Lead Noise Alert™ was not analyzed.

Twelve leads were subjected to continuous, cyclic bending in a fatigue tester (Bose Model 3230, Eden Prairie, Minn.). Each lead was clamped to a fixed lower plate and a movable upper plate. The upper plate was attached to a fatigue-test frame that bent a 1.25 cm lead segment between the clamps in a “U” shape to a minimum bending radius of 0.15 cm. The lead was oriented with the helix on the inner radius to place greater stress on the helix than the cables and thus ensure helix fracture. To measure DCR directly, separate electrical connections were made to the helix conductor proximal and distal to the flexing portion.

The lead and generator were placed in a saline bath, except for the proximal connection to the helix conductor. A 1 Hz (1000 ms interval), simulated ECG signal was applied to the saline solution using patch electrodes. The lead's tip-ring sensing electrodes were oriented approximately perpendicular to the patch electrodes to ensure that the ICD sensed the simulated ECG. The receiving-coil of a Holter monitor was positioned on the generator to record ICD EGMs continuously.

The fatigue tester was programmed for sinusoidal motion at 1.3 Hz (769 ms interval). The maximum and minimum bending radii of 0.15 cm and 0.5 cm were preselected to cause complete fracture of the helix in 10,000-100,000 bending cycles (12). Custom LabVIEW® software ended testing based on a DCR criterion corresponding to complete fracture (see below). The test ended within 3 minutes after this criterion was met. At test end, high resolution industrial radiographs (Northstar M50, Rogers, Minn.) were performed for each lead at the minimum and maximum bending radii.

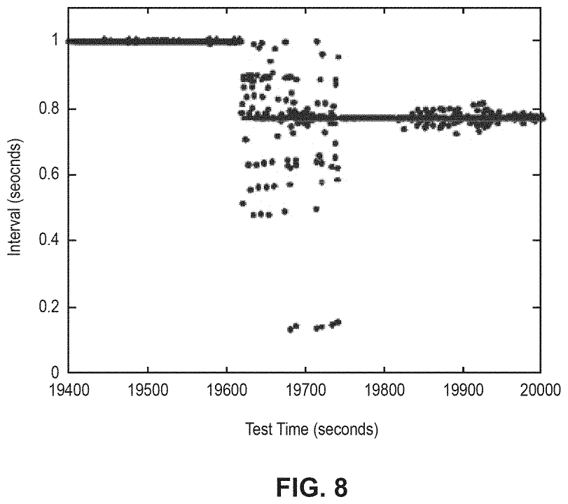

Both ICD stored EGMs and those telemetered continuously and recorded by the Holter were analyzed. Both comprised the tip-ring sensing channel, Can-RV coil shock channel, and ventricular marker channel. FIG. 8 illustrates V-V intervals over time during testing. The first oversensed event was defined as the first V-V interval <1000 ms on the Holter marker channel. All other EGM events were determined by the ICD.

DCR was sampled at 65 Hz for 7.7 seconds (500 samples) every 3 minutes using a meter with range 10⁻⁵-10³⁸Ω (Agilent model 3458A) controlled by a custom LabVIEW program. The standard deviation of DCR (s_{DCR}) was calculated in near real time. Each set of 500 DCR samples was written to a file with time stamps for subsequent analysis. A combination of specialized ICD programming and custom LabVIEW® software was used to correlate time markers for ICD stored events, telemetered Holter EGMs, and DCR measurements.

Resistance Criteria for Partial and Complete Fracture.

Criteria for partial and complete fracture were needed to determine when to interrupt or stop testing. These criteria were defined using DCR, which was monitored by controlling software every 3 minutes, rather than EGMs and intervals which were not monitored in real time by the controller.

Partial fracture was defined as s_{DCR}≤0.5Ω. This criterion was based on the data from pilot testing in air and preliminary recordings made in saline that showed a maximum baseline s_{DCR} of 0.05Ω. We set the partial fracture criterion to be 10 times this maximum baseline value. This criterion was evaluated for 5 leads in the present study: When s_{DCR} first exceeded 0.5Ω, fatigue cycling was stopped, ICD arrhythmia detection was suspended, the Holter recorder was stopped, and the lead was disconnected from the generator for imaging. Partial fracture was identified radiographically by discontinuity of at least 1 filar. Two leads were returned to the test apparatus after imaging and tested to complete fracture.

Complete fracture was defined as DCR≥3000Ω, based on radiographs illustrated in FIG. 7. Complete fracture was identified radiographically by discontinuity of all 4 filars. In the present study, 9 leads were tested until this criterion was met and then imaged.

Correlation of Experimental Resistance with Clinical Pacing Impedance.

For the purpose of relating the measured DCR to ICD system-measured pacing impedance, the distribution of pacing impedances measured 6 months after implant in 8139 patients with comparable, functioning RV defibrillation leads followed in the CareLink® remote monitoring system were analyzed. The median, 10^{th} percentile, and 90^{th} percentile of the clinical impedance distribution to determine the DCR at which clinical alerts would occur for high pacing impedance and for LIA's relative-impedance criterion was calculated.

Events defined by DCR or clinical impedance comprised partial fracture (s_{SCR}≥0.5Ω), LIA relative-impedance criterion (≥75% increases from baseline), nominal impedance alert (>2000Ω), and DCR>3000Ω. Events defined by EGM characteristics included first oversensing, Sensing Integrity Count ≥30, first and second stored fast NSTs, LIA triggered by both oversensing criteria, and first inappropriate detection of VF. Since EGMs were recorded continuously and DCR was recorded every 3 minutes, events defined by EGMs with the DCR record in closest temporal proximity were correlated. Additionally, Holter EGMs at each 20^{th} percentile of total time from partial fracture (T_{PF}) to complete fracture (T_{CF}) were inspected. The time at which events occurred was normalized as a multiple of T_{PF}.

Median times to analyzed event were compared using the Wilcoxon signed rank test. The Bonferroni method was used to adjust p-values to correct for multiple comparisons. A p-value <0.05 was considered significant.

For all 12 leads, baseline DCR was 41±2.8Ω and baseline s_{DCR} was ≤0.05Ω. Differences among leads were due to variation in contact resistance at connections to the measuring apparatus.

For 8139 functioning, implanted leads, the median pacing impedance at 6 months was 446Ω, 10^{th} percentile 361Ω, and 90^{th} percentile 560Ω. Using median values, the LIA relative-impedance criterion (>75% increase) corresponded to a DCR of 375Ω; the nominal impedance alert (2000Ω) corresponded to a DCR of 1594Ω.

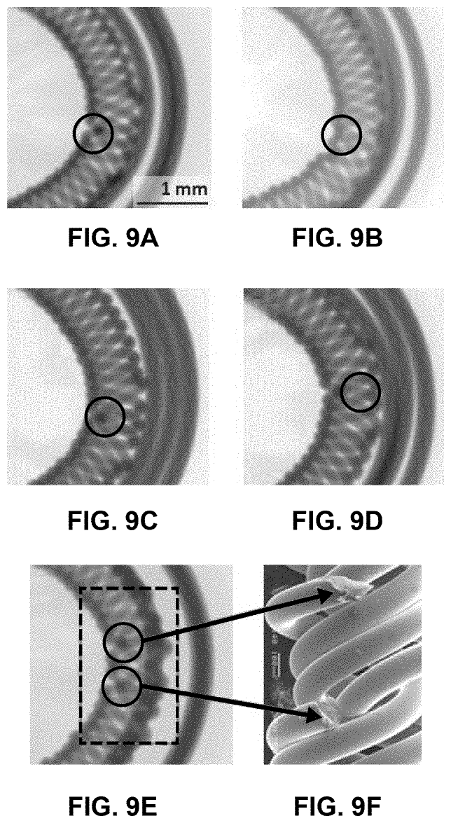

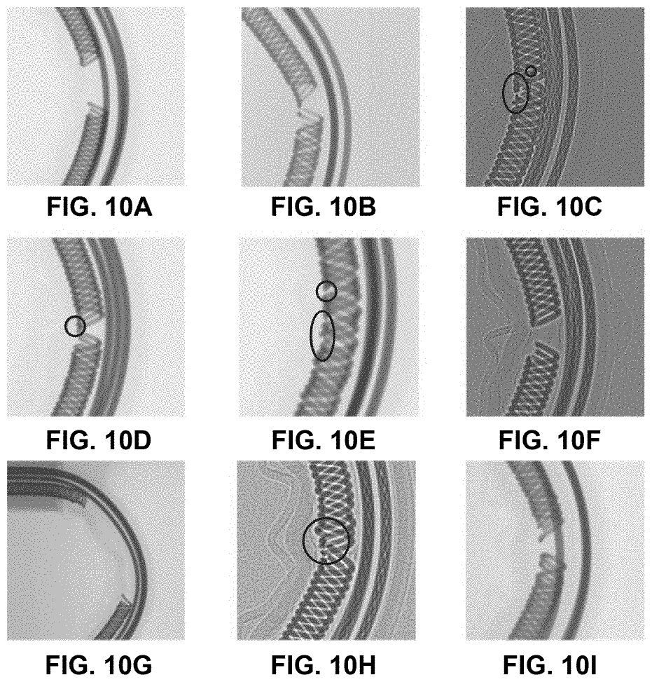

FIG. 9 illustrates radiographs of five leads imaged when the DCR-based partial fracture criterion was met (s_{DCR}≥0.5Ω), which showed 1 or 2 fractured helix filars near the apex of the bend. FIG. 10 illustrates radiographs of all nine leads imaged when the DCR-based complete fracture criterion was met (DCR≥3000Ω), which confirmed fracture of all 4 filars.

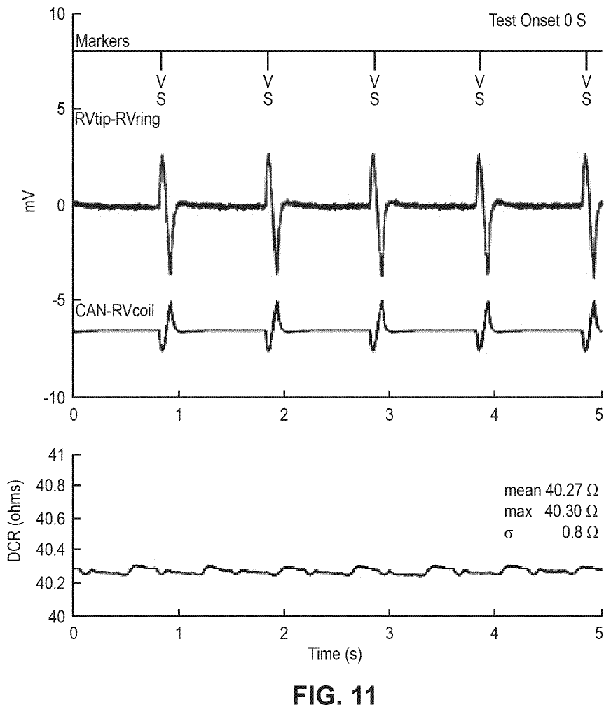

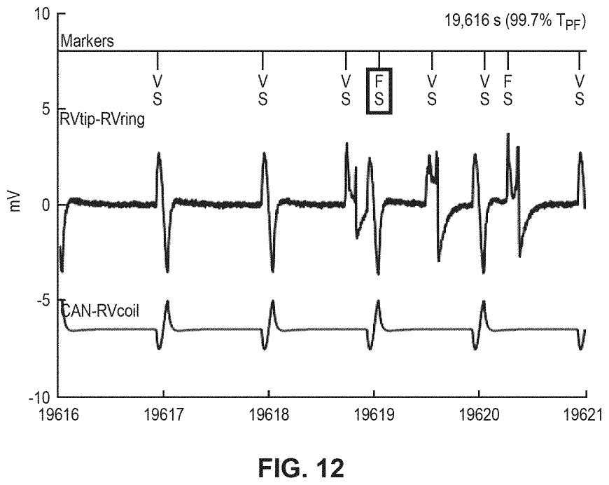

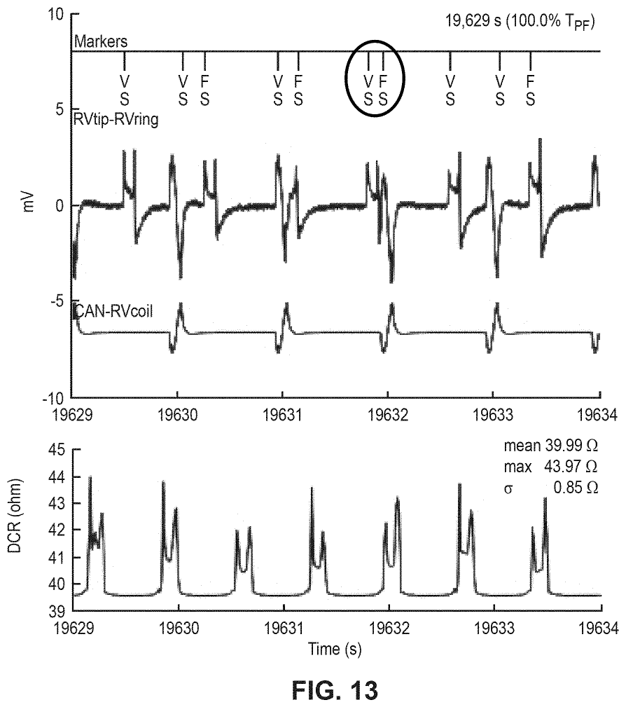

FIGS. 11-17 shows the sequence of corresponding DCR and EGM changes in a representative lead. FIG. 12 illustrates that initial oversensed signals occur at the bending frequency. FIG. 13 illustrates that the first DCR sample taken 13 seconds later meets the s_{DCR} criterion for partial fracture. The corresponding EGM shows the first non-physiologic short interval. For each bending cycle, there is one fracture-induced, double-peak electrical signal and one corresponding double peak DCR spike.

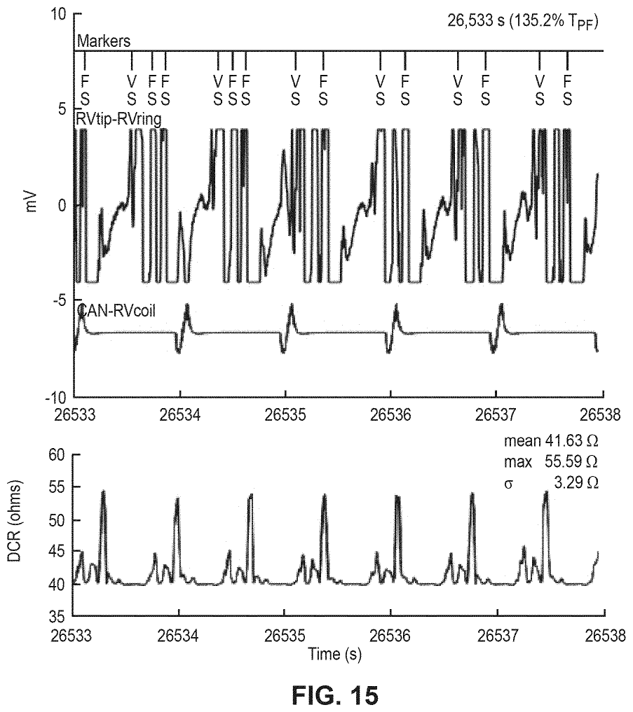

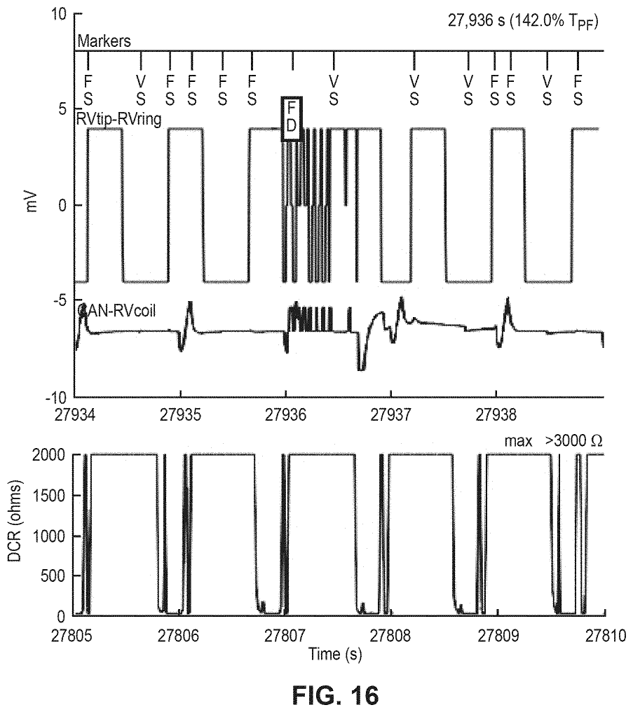

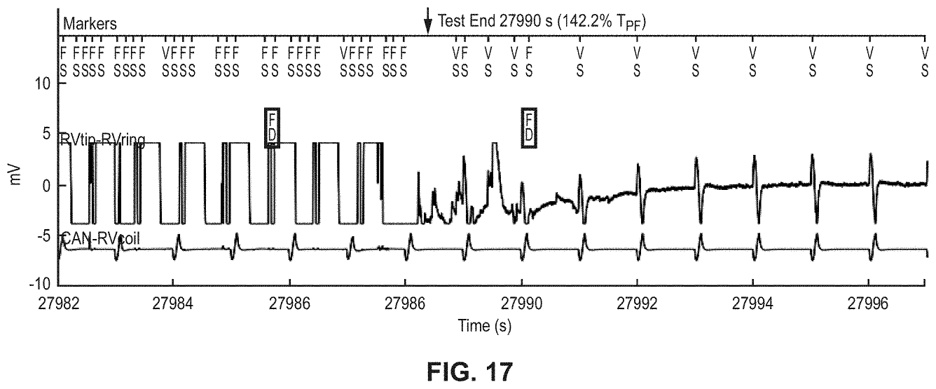

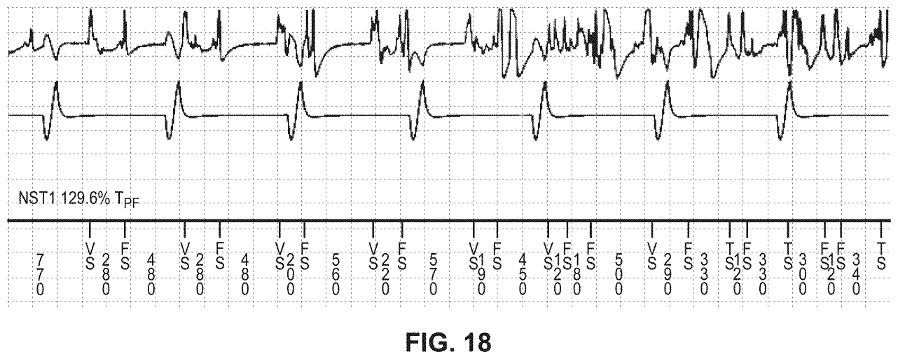

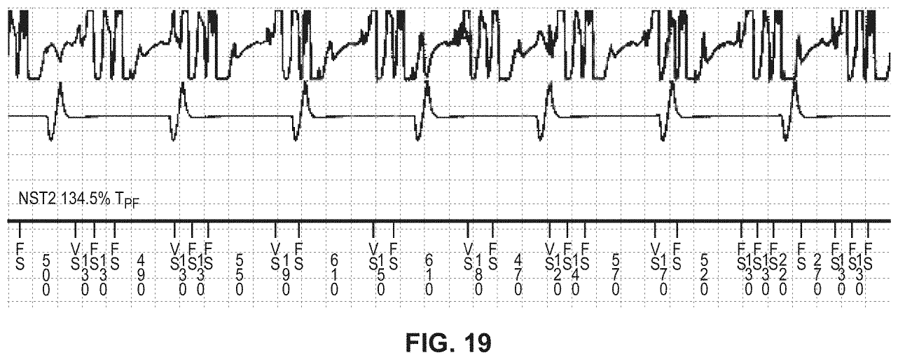

FIG. 15 shows that the first, high-rate NST occurs when a second oversensed signal occurs per bending cycle, corresponding to a second DCR spike. FIG. 15 corresponds to triggering of LIA by both oversensing components when the second NST (shown in FIG. 18) was recorded within 1 second of the SIC reaching the threshold value of 30. Longer bursts of oversensed signals saturate the sense amplifier. FIG. 16 shows the first inappropriate detection of VF. The relative-impedance criterion, impedance-threshold alert, and complete fracture criterion were triggered simultaneously, 2 minutes earlier. FIG. 17 shows that oversensing stops and the EGM normalizes when bending ceases at test end. FIGS. 18 and 19 show EGM and marker channel data during bending testing of a lead.

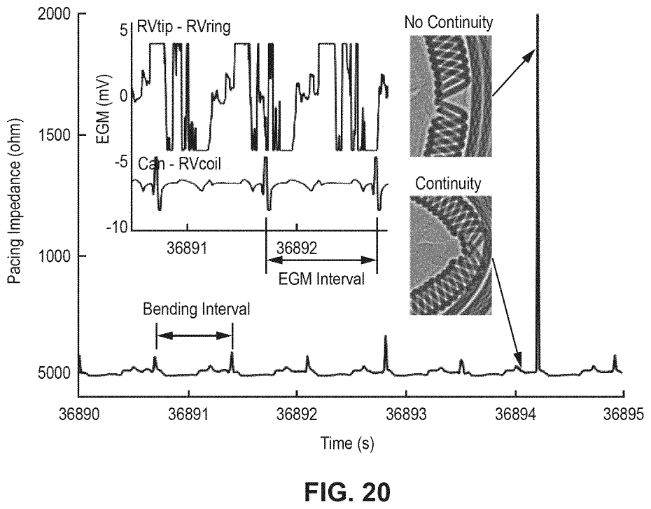

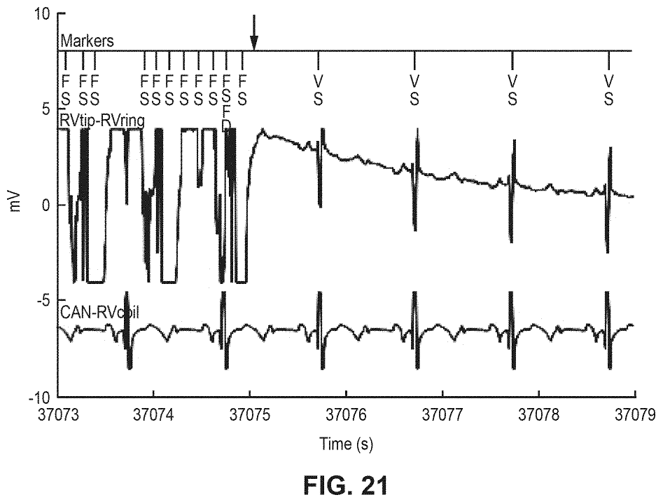

FIGS. 20 and 21 show that the relative-impedance criterion, impedance-threshold alert, and complete fracture criterion are triggered simultaneously by a DCR spike synchronized to the bending cycle. Yet the baseline DCR increased by less than 10Ω; and all values except the designated spike are within the normal range; so, a single clinical impedance measurement would likely be normal. Radiograph shows overlapping filar ends at minimum bending radius, explaining how electrical continuity is preserved despite complete fracture. The EGM normalizes as the bending stops.

Table 1 shows the time to events defined by EGMs or DCR in all 9 leads tested to complete fracture. The events include non-sustained tachycardia (NST), Medtronic Lead Integrity Alert (LIA), and ventricular fibrillation (VF).

TABLE 1

| TIME TO EVENT (minutes) | | | | | | |
|---|---|---|---|---|---|---|
| | Partial | 1st | 1st High | LIA | 1st | Complete |
| Lead | Fracture | Oversensing | Rate NST | alert | VF | Fracture |
| A | 333 | 334 | 334 | 361 | 387 | 467 |
| B | 212 | 209 | 242 | 243 | 294 | 2014 |
| C | 451 | 419 | 498 | 510 | 555 | 580 |
| D | 370 | 372 | 422 | 470 | 471 | 615 |
| E | 309 | 311 | 314 | 315 | 332 | 500 |
| F | 224 | 227 | 227 | 234 | 227 | 272 |
| G | 536 | 533 | 533 | 540 | 1096 | 1093 |
| H | 382 | 384 | 485 | 518 | 519 | 872 |
| I | 328 | 327 | 425 | 441 | 466 | 464 |

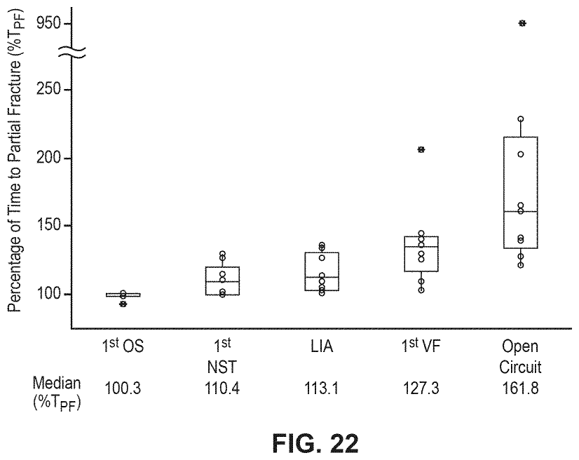

Table 2 and FIG. 22 display times normalized to T_{PF}. Median T_{PF} and T_{CF} were 334 min and 580 minutes, respectively. Since DCR was measured every 3 min, intervals between DCR measurements correspond approximately to a median of 1% T_{PF}.

TABLE 2

| NORMALIZED TIME TO EVENT | | | | | |
|---|---|---|---|---|---|
| | 1st | 1st High | LIA | 1st | Complete |
| Lead | Oversensing | Rate NST | alert | VF | Fracture |
| A | 100.3 | 100.3 | 108.4 | 116.2 | 140.2 |
| B | 98.6 | 114.2 | 114.6 | 138.7 | 950.0 |
| C | 92.8 | 110.4 | 113.1 | 123.1 | 128.6 |
| D | 100.5 | 114.1 | 127.0 | 127.3 | 166.2 |
| E | 100.8 | 101.6 | 101.9 | 107.4 | 161.8 |
| F | 101.2 | 101.2 | 104.4 | 101.2 | 121.4 |
| G | 99.5 | 99.4 | 100.7 | 204.5 | 203.9 |
| H | 100.6 | 127.0 | 135.6 | 135.9 | 228.3 |
| I | 99.7 | 129.6 | 134.5 | 142.1 | 141.5 |

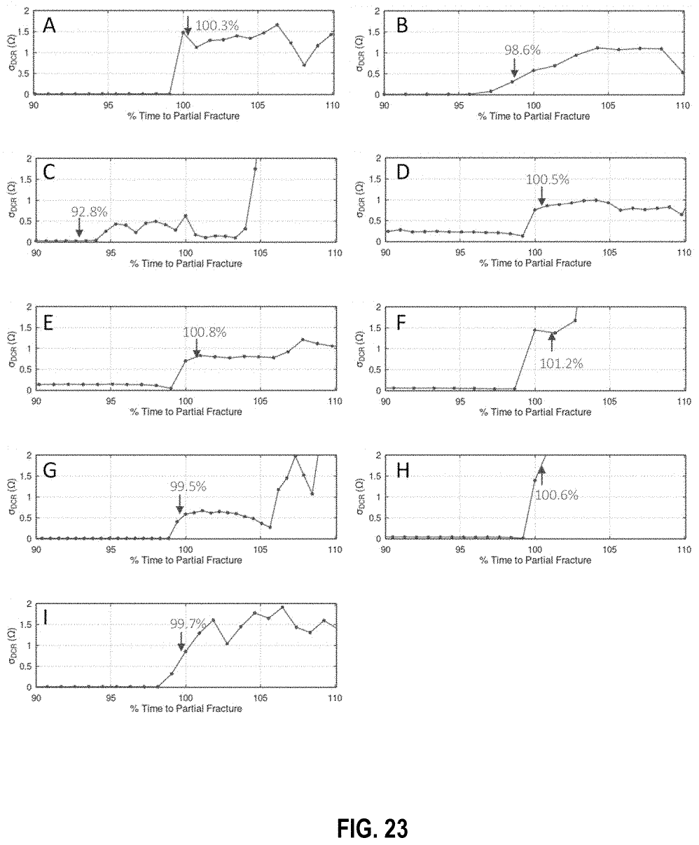

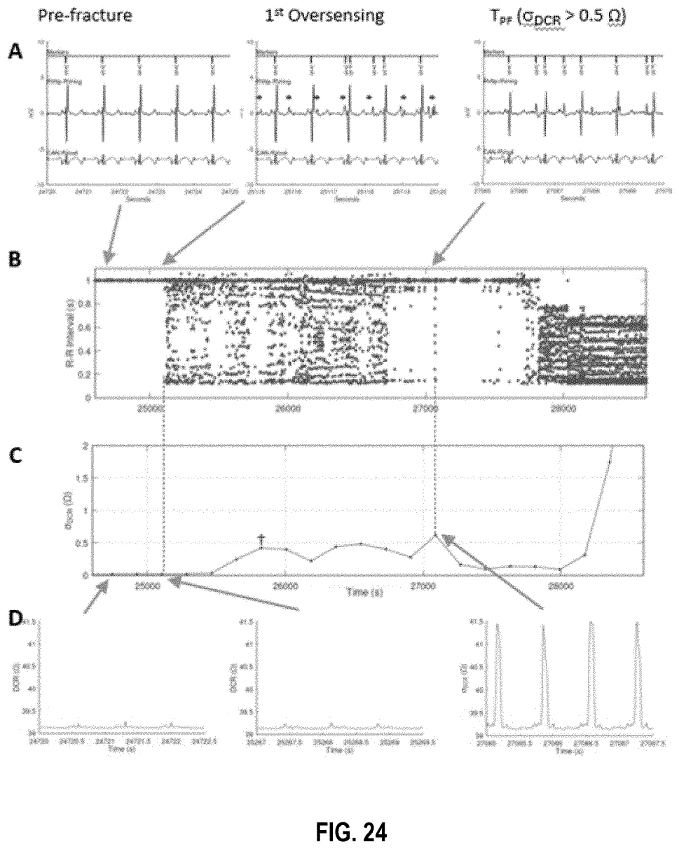

At T_{PF}, the peak DCR was 45±2.2Ω and s_{DCR} 0.97±0.40Ω. The first oversensed event occurred at a median of 100.3% T_{PF}, within 1.4% of T_{PF} in 8 of 9 leads, as illustrated respectively by panels A-I in FIG. 23. Panel C of FIG. 23 illustrates that, in the remaining lead, the first oversensed event occurred at 92.9% T_{PF}. The timing of the first non-physiologic short interval varied from simultaneous with the first oversensed event to 96 s later. FIG. 24 shows details of the outlier lead in Panel C of FIG. 23.

LIA was triggered by both oversensing criteria at 113% T_{PF}, before detection of VF in 8 of 9 leads (p<0.006). In the remaining lead, the first repetitive oversensing continued to detection of VF, so a second NST was not recorded before VF detection. In contrast, the LIA relative-impedance criterion and impedance-threshold alert were triggered after VF detection in 7 of 9 leads (p=0.03), and 0.6% T_{PF} before detection in the remaining 2 leads. Neither clinical impedance diagnostic was triggered before complete fracture (162% T_{PF}) in any lead. In all leads, impedance alerts occurred in the last set of DCR samples, regardless of whether we used median, 10^{th} percentile, or 90^{th} percentile of the clinical impedance distribution.

DCR deviations from baseline occurred only with bending until complete fracture, but they increased in amplitude and complexity as the fracture progressed. Even after complete fracture, DCR varied with compression and retained at least a short isoelectric baseline in 8 of 9 leads.

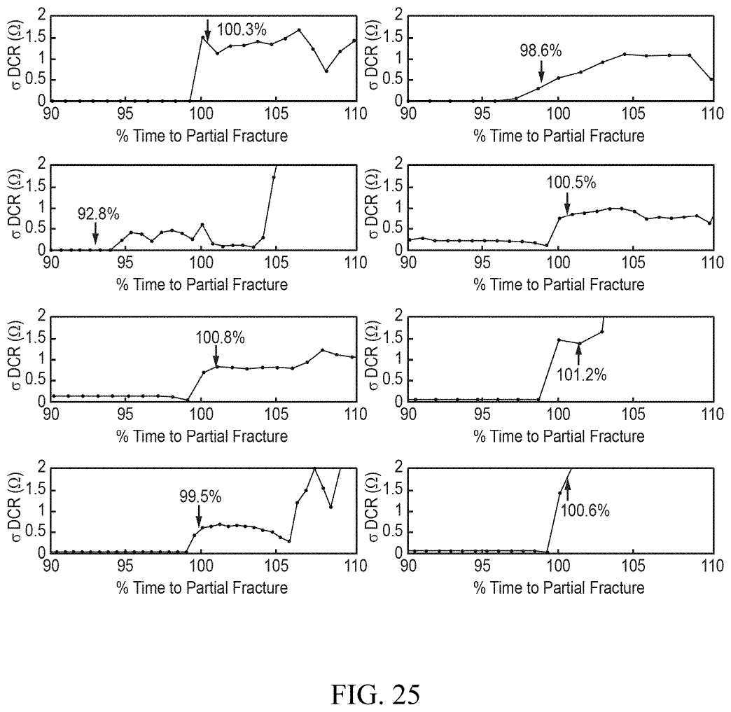

Fracture-induced signals had some common characteristics across leads, as illustrated in FIG. 25, but no two leads showed identical patterns: (1) The first signals were discrete, usually including one or more high-frequency components, and usually occurred once per bending cycle. (2) Device-detected NST correlated with the onset of multiple oversensed events per cycle. (3) At complete fracture, all leads had continuous or near-continuous oversensing. (4) Signal truncation caused by sensing-amplifier saturation became more likely as the fracture progressed, occurring in only 1 of the first oversensed signals but in all complete fractures.

In all early fractures, oversensed events correlated closely with small, cyclical increases in DCR (median peak increase 4Ω). With progression, cyclical fracture-induced signals became longer than DCR deviations from baseline.

In-vitro bending tests monitor DCR to determine fatigue and fracture properties of pacing conductors, providing an estimate of the service life of leads under expected use conditions of stress and rate of cyclic bending (13). Usually, testing is performed on short segments of conductors rather than complete leads, continued to complete fracture so the onset of partial fracture is not determined, and performed in air so fracture-induced signals cannot be recorded. In this study, a complete defibrillation lead in a saline tank connected to an ICD generator was tested. This may be the first study to determine the sequences of EGM and DCR/impedance changes in developing conductor fracture and correlate them, both with each other and with radiographs of partial fracture.

Changes in Resistance/Pacing Impedance.

These tests demonstrate that partial fracture occurs with an increase in s_{DCR}<0.5Ω. Until complete fracture, DCR deviations from baseline occur only intermittently, with bending. A likely explanation is that DCR spikes occur when the fracture faces of individual filars lose contact at specific phase(s) of the bending cycle. In other phases, the lead body constrains the helix so that fracture faces appose, preserving electrical continuity.

The experimental fractures reproduce several characteristic features of oversensing in clinical fractures: intermittent occurrence, non-physiologic short intervals, and highly-variable EGMs with both high-frequency components and high-amplitude components that saturate the sensing amplifier.

The tests also demonstrate that oversensed signals in early fractures correspond to bending-induced changes in DCR. EGMs normalize when bending stops, even after complete fracture. These observations provide direct evidence that make-break potentials (15) caused by intermittent contact between fracture faces are responsible for fracture-induced oversensing.

Although small changes in DCR are highly-sensitive for fracture in fatigue testing of conductor segments, clinical oversensing alerts are more sensitive than impedance alerts. In a report of lead failures from multiple manufacturers, 88% of LIAs were triggered by both oversensing criteria vs. 12% by one oversensing and one impedance criterion.

The findings of this experiment explain both discrepancies in sensitivity, between DCR measured in-vitro vs. impedance measured clinically and between clinical oversensing diagnostics vs. impedance measurements. Early conductor fractures cause both fracture-induced signals and small, but consistent, increases in peak DCR and s_{DCR}. The fracture-induced signals can be detected by clinical oversensing diagnostics, but the DCR increases are too small to be detected by ICD impedance diagnostics. In this study, ICD oversensing alerts always were triggered by partial fractures, but impedance alerts never were triggered until complete fracture occurred. Additionally, ICDs monitor for oversensing continuously; but they measure impedance only intermittently. Because most fractures in modern leads occur in the shoulder region, fracture detection depends on skeletal muscle activity to initiate motion at the fracture site. Unless the ICD measures impedance serendipitously during muscle activity, the measurement will be normal in partial fractures and even in some complete fractures. Complete fracture confirmed by returned-product analysis has been reported with a normal impedance trend.

While only one manufacturer's diagnostics and one lead model were tested, these results likely apply to leads and diagnostics from other manufacturers: The mechanism of fracture in this experiment is mechanically consistent with that of helix-conductor fractures in leads from multiple manufacturers, and both DCR values and electrical potentials responsible for oversensing are determined by universal laws of physics.

Further, oversensing diagnostics have similarities across manufacturers. In this experiment, non-physiologic-short intervals begin nearly simultaneously with the first fracture-induced signals. The median time to the threshold count (30) for Medtronic's SIC was approximately equal to that for occurrence of two rapid NSTs caused by oversensing, triggering LIA. Thus, although clinical sensitivity has not been reported for these two diagnostics, each is likely to be approximately as sensitive as LIA for detecting conductor fractures.

This study's primary limitation is that it does not fully reproduce the environment of clinical fractures. In lead bending, the minimum radius is inversely proportional to applied stress. In our study, the minimum radius (0.15 cm) was slightly less than the smallest radius measured in the only reported biplane fluoroscopic analysis of clinical lead bending (0.18 cm) (18); but it was within that study's 95% confidence interval for a 5^{th} percentile bending radius. The greater experimental stress may have caused more separation of coil filars in complete fracture than usually occurs in clinical fractures. Either this difference or our small sample size could explain extended periods with clinical impedance in the range 1000-2000Ω, as occurs in approximately 10% of clinical fractures (3-5,9), were not observed. In modern leads, most fractures occur near the anchor sleeve or under the clavicle (16) due to intermittent and varying bending stress, over a period of years. In contrast, cyclic stress was applied at a constant amplitude and frequency selected to cause complete fracture in a practical, experimental time frame of less than 12 hours. Continuous bending may have resulted in more rapid accumulation of oversensed intervals in the VF zone than usually occurs with intermittent clinical bending.

Oversensing occurs at the earliest DCR and radiographic signs of partial conductor fracture. In contrast, clinical impedance alerts are only triggered by complete fracture. In early fractures, fracture-induced signals and DCR/impedance increases occur simultaneously, during lead bending. When bending stops, EGMs return to baseline. These findings provide strong evidence that fracture-induced signals are caused by make-break potentials. They also suggest opportunities for improving lead diagnostics.

Various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processing circuitry” may generally refer to any of the foregoing logic circuitry, alone or in combination with other logic circuitry, or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

The following examples are illustrative of the techniques described herein.

Example 1: A method comprising: acquiring a set of measurements of impedance of an implantable medical lead; determining a metric of variability of the set of impedance measurements; determining that the metric of variability satisfies a criterion; and generating a lead integrity alert in response to the metric of variability satisfying the criterion.

Example 2: The method of example 1, wherein the set of measurements comprises impedances measured at a rate of N samples per second, wherein N is an integer greater than or equal to 1.

Example 3: The method of example 2, wherein N equals 65.

Example 4: The method of any of examples 1 to 3, wherein set of measurements comprises 500 measurements.

Example 5. The method of any of examples 1 to 4, wherein a resolution of the measurements is less than or equal to 0.1 ohms.

Example 6: The method of any of examples 1 to 5, wherein the metric of variability comprises a standard deviation of the set of measurements.

Example 7: The method of any of examples 1 to 6, wherein determining that the metric of variability satisfies the criterion comprises determining that the metric of variability is greater than or equal to a threshold impedance value.

Example 8: The method of any of examples 1 to 7, wherein acquiring the set of measurements of impedance of the implantable medical lead comprises acquiring a set of measurements of a path including at least one electrode of the implantable medical lead.

Example 9: The method of example 8, wherein generating the lead integrity alert comprises indicating at least one of a fracture, partial fracture, or anticipated fracture of a conductor connector to the electrode.

Example 10: The method of any of examples 1 to 9, wherein the implantable medical lead comprises an intracardiac lead.

Example 11: A system comprising: an implantable medical device configured to measure impedance of an implantable medical lead coupled to the implantable medical device; and processing circuitry configured to perform the method of any of examples 1 to 10.

Example 12: The system of example 11, wherein the processing circuitry comprises processing circuitry of the implantable medical device.

Example 13: The system of example 11 or 12, further comprising a computing device configured to wirelessly communicate with the implantable medical device, wherein the processing circuitry comprises processing circuitry of the computing device.

Example 14: The system of example 11 or 12, further comprising a computing device configured to wirelessly communicate with the implantable medical device, wherein the computing device is configured to present the lead integrity alert to a user.

Example 15: A non-transitory computer-readable storage medium comprising instructions that, when executing by processing circuitry, cause the processing circuitry to perform the method of any of examples 1 to 10.

Example 16: A system comprising means to perform the method of any of examples 1 to 10.

What is claimed is:

1. A method comprising:
acquiring a set of measurements of impedance of an implantable medical lead;
determining a metric of variability of the set of impedance measurements;
determining that the metric of variability satisfies a criterion; and
generating a lead integrity alert in response to determining that the metric of variability satisfies the criterion.

2. The method of claim 1, wherein the set of measurements comprises impedances measured at a rate of N samples per second, wherein N is an integer greater than or equal to 1.

3. The method of claim 2, wherein N equals 65.

4. The method of claim 1, wherein set of measurements comprises 500 measurements.

5. The method of claim 1, wherein a resolution of the measurements is less than or equal to 0.1 ohms.

6. The method of claim 1, wherein the metric of variability comprises a standard deviation of the set of measurements.

7. The method of claim 1, wherein determining that the metric of variability satisfies the criterion comprises determining that the metric of variability is greater than or equal to a threshold impedance value.

8. The method of claim 1, wherein acquiring the set of measurements of impedance of the implantable medical lead comprises acquiring a set of measurements of a path including at least one electrode of the implantable medical lead.

9. The method of claim 8, wherein generating the lead integrity alert comprises indicating at least one of a fracture, partial fracture, or anticipated fracture of a conductor connector to the at least one electrode.

10. The method of claim 1, wherein the implantable medical lead comprises an intracardiac lead.

11. A system comprising:
an implantable medical device configured to measure impedance of an implantable medical lead coupled to the implantable medical device; and
processing circuitry configured to:
acquire a set of measurements of impedance of the implantable medical lead by the implantable medical device;
determine a metric of variability of the set of impedance measurements;
determine that the metric of variability satisfies a criterion; and
generate a lead integrity alert in response to determining that the metric of variability satisfies the criterion.

12. The system of claim 11, wherein the set of measurements comprises impedances measured at a rate of N samples per second, wherein N is an integer greater than or equal to 1.

13. The system of claim 12, wherein N equals 65.

14. The system of claim 11, wherein set of measurements comprises 500 measurements.

15. The system of claim 11, wherein a resolution of the measurements is less than or equal to 0.1 ohms.

16. The system of claim 11, wherein the metric of variability comprises a standard deviation of the set of measurements.

17. The system of claim 11, wherein, to determine that the metric of variability satisfies the criterion, the processing circuitry is configured to determine that the metric of variability is greater than or equal to a threshold impedance value.

18. The system of claim 11, wherein, to acquire the set of measurements of impedance of the implantable medical lead, the processing circuitry is configured to acquire a set of measurements of a path including at least one electrode of the implantable medical lead.

19. The system of claim 18, wherein, to generate the lead integrity alert, the processing circuitry is configured to indicate at least one of a fracture, partial fracture, or anticipated fracture of a conductor connector to the at least one electrode.

20. The system of claim 11, wherein the implantable medical lead comprises an intracardiac lead.

21. The system of claim 11, wherein the processing circuitry comprises processing circuitry of the implantable medical device.

22. The system of claim 11, further comprising a computing device configured to wirelessly communicate with the implantable medical device, wherein the processing circuitry comprises processing circuitry of the computing device.

23. The system of claim 11, further comprising a computing device configured to wirelessly communicate with the implantable medical device, wherein the computing device is configured to present the lead integrity alert to a user.

24. A non-transitory computer-readable storage medium comprising instructions that, when executing by processing circuitry, cause the processing circuitry to:
acquire a set of measurements of impedance of the implantable medical lead by the implantable medical device;
determine a metric of variability of the set of impedance measurements;
determine that the metric of variability satisfies a criterion; and
generate a lead integrity alert in response to determining that the metric of variability satisfies the criterion.
